(19) United States
(12) Patent Application Publication
(10) Pub. No.: US 2024/0294496 A1
(43) Pub. Date: Sep. 5, 2024

(54) PRODUCTION METHOD FOR SYNTHETIC INTERMEDIATE OF MONOCYCLIC PYRIDINE DERIVATIVE

(71) Applicant: Eisai R&D Management Co., Ltd., Bunkyo-ku, Tokyo (JP)

(72) Inventors: Mitsuo NAGAI, Kamisu-shi, Ibaraki (JP); Takashi FUKUYAMA, Tsukuba-shi, Ibaraki (JP); Yasuaki KAMADA, Shinagawa-ku, Tokyo (JP); Jun NIIJIMA, Tsukuba-shi, Ibaraki (JP); Hirofumi KURODA, Kamisu-shi, Ibaraki (JP); Yuki KAROJI, Kamisu-shi, Ibaraki (JP); Keiichi MURAKAMI, Kamisu-shi, Ibaraki (JP); Masayuki OMORI, Kamisu-shi, Ibaraki (JP); Yusuke MIYASHITA, Kamisu-shi, Ibaraki (JP); Atsushi KAMADA, Tsukuba-shi, Ibaraki (JP); Masaaki MATSUDA, Tsukuba-shi, Ibaraki (JP)

(21) Appl. No.: 18/292,731

(22) PCT Filed: Aug. 29, 2022
(86) PCT No.: PCT/JP2022/032309

§ 371 Date: Jan. 26, 2024

(30) Foreign Application Priority Data
Aug. 31, 2021 (JP) 2021-141245

(51) Int. Cl. C07D 401/12; C07C 201/12; C07C 253/30; C07D 209/08

(52) U.S. Cl. CPC C07D 401/12; C07C 201/12; C07C 253/30; C07D 209/08

(57) ABSTRACT

The invention provides a production process allowing more high-yield and efficient synthesis of key intermediates (compound (2i) or a salt thereof, and compound (1g)) for production of E7090, which is useful as an FGFR inhibitor.

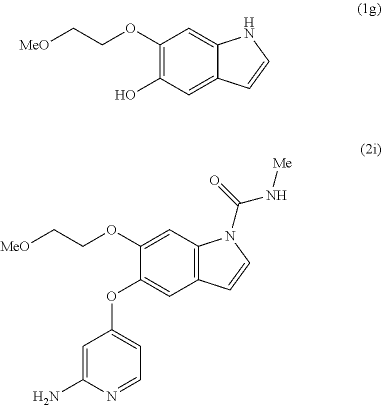 [SMILES: COCCOc1cc2[nH]ccc2cc1O ; CNC(=O)n1ccc2cc(Oc3ccnc(N)c3)c(OCCOC)cc21]

### TECHNICAL FIELD

The present invention relates to a method for producing synthesis intermediates of monocyclic pyridine derivatives that are useful as FGFR inhibitors.

### BACKGROUND ART

The monocyclic pyridine derivative 5-((2-(4-(1-(2-hydroxyethyl)piperidin-4-yl)benzamide)pyridin-4-yl)oxy)-6-(2-methoxyethoxy)-N-methyl-1H-indole-1-carboxamide butanedioate (2:3)

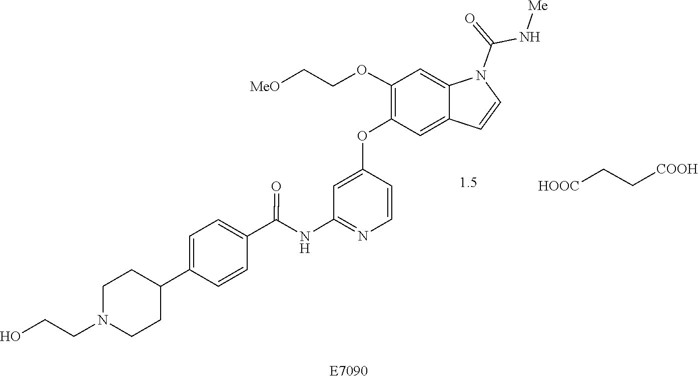 [SMILES: CNC(=O)n1ccc2cc(Oc3ccnc(NC(=O)c4ccc(C5CCN(CCO)CC5)cc4)c3)c(OCCOC)cc21 ; O=C(O)CCC(=O)O]

(hereunder also referred to as “E7090”) has potent FGFR (Fibroblast Growth Factor Receptor) inhibitory action and is useful as a therapeutic agent for FGFR kinase-associated intrahepatic cholangiocarcinoma and breast cancer (PTLs 1 and 2).

### CITATION LIST

### Patent Literature

[PTL 1] International Patent Publication No. WO2014/129477
[PTL 2] International Patent Publication No. WO2016/027781

### SUMMARY OF INVENTION

### Technical Problem

Methods for producing important intermediates of E7090 are described in PTL 1 (Examples 20 and 22) and PTL 2 (Example 1).

Compound (1g) in PTL 1 is synthesized by reacting 1-bromo-3-methoxypropane with 3,4-dihydroxybenzaldehyde (compound (P 1-1)), but the selectivity for alkylation of the phenolic hydroxyl group is low, while highly toxic and hazardous nitromethane is used for conversion from compound (P 1-3) to compound (P 1-4), and compound (P 1-5) obtained by nitration of compound (P 1-4) is an explosive compound. Moreover, the steps for converting compound (P 1-3) to compound (P 1-5) via compound (P 1-4) are all highly dangerous. It has therefore been desirable to devise a new, safe and efficient route of synthesis that produces higher yield.

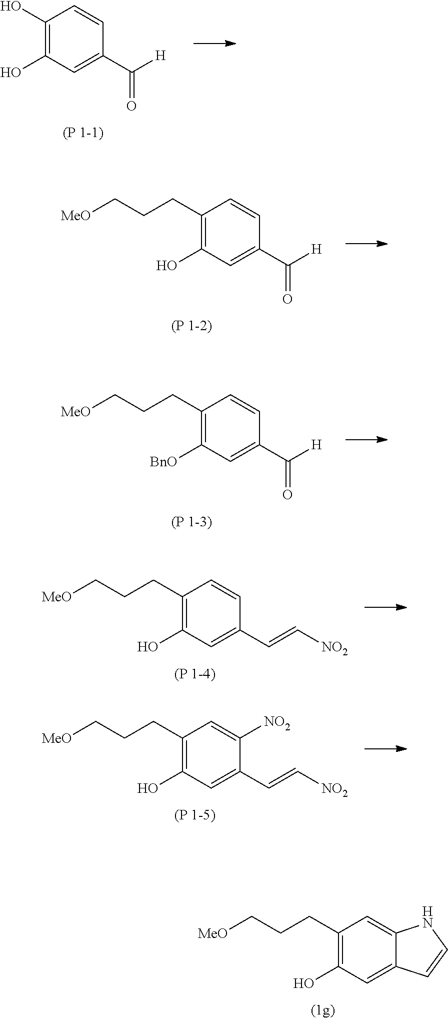 [SMILES: O=Cc1ccc(O)c(O)c1 ; COCCCc1ccc(C=O)cc1O ; COCCCc1ccc(C=O)cc1OCc1ccccc1 ; COCCCc1ccc(/C=C/[N+](=O)[O-])cc1O ; COCCCc1cc([N+](=O)[O-])c(/C=C/[N+](=O)[O-])cc1O ; COCCCc1cc2[nH]ccc2cc1O]

Compound (2i) described in PTL 1 is synthesized by reacting 4-chloropyridine (compound (P 2-1)) with compound (1g) in the presence of a base, but the yield of the target compound (P 2-2) has been low since N,O-diallylated products are also produced in addition to compound (P 2-2), due to allylation of the indole nitrogen atom together with the phenolic hydroxyl group. The reaction also requires a high temperature of 150° C. or above, and therefore a method has been sought for constructing heterobiaryl backbones under mild conditions.

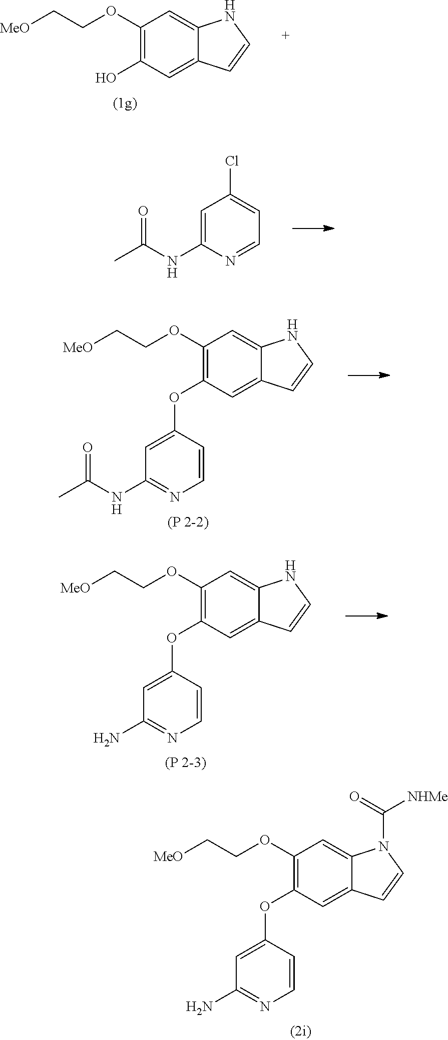 [SMILES: COCCOc1cc2[nH]ccc2cc1O ; CC(=O)Nc1cc(Cl)ccn1 ; COCCOc1cc2[nH]ccc2cc1Oc1ccnc(NC(C)=O)c1 ; COCCOc1cc2[nH]ccc2cc1Oc1ccnc(N)c1 ; CNC(=O)n1ccc2cc(Oc3ccnc(N)c3)c(OCCOC)cc21]

It is an object of the present application to provide a production process allowing high-yield and efficient synthesis of key intermediates for production of E7090, which is useful as an FGFR inhibitor.

### Solution to Problem

Provided herein are synthesis intermediates that are useful for production of E7090, which is useful as an FGFR inhibitor, as well as a process for their production. Specifically, the present invention provides the following [1] to [7].
[1] A process for producing compound (2i):

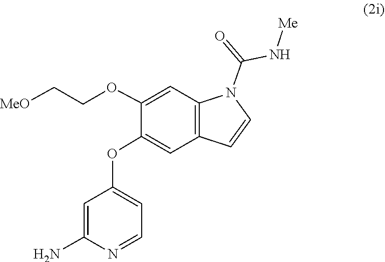 [SMILES: CNC(=O)n1ccc2cc(Oc3ccnc(N)c3)c(OCCOC)cc21]

or a salt thereof, wherein the process comprises:
2-a) step 2-a) in which a protecting group is introduced into compound (1g):

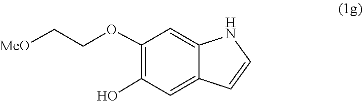 [SMILES: COCCOc1cc2[nH]ccc2cc1O]

to produce compound (2a):

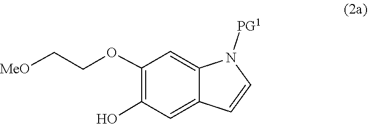

(where PG¹ represents a nitrogen protecting group),
2-b) step 2-b) in which compound (2a) obtained in step 2-a) and compound (2b):

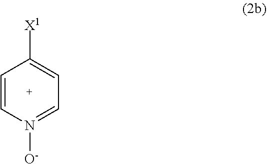

(where X¹ represents a leaving group),
are reacted in the presence of a base to produce compound (2c):

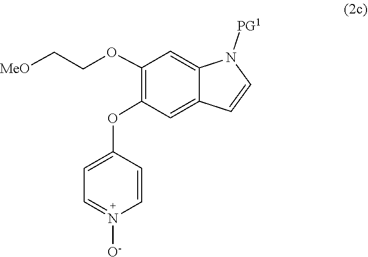

(where PG¹ represents the same group as specified above),
2-c) step 2-c) in which compound (2c) obtained in step 2-b) and compound (2d):

R¹—NH₂ (2d)
(where R¹ represents a tert-pentyl, tert-butyl, tert-octyl or cumyl group),
are reacted in the presence of an activating agent to produce compound (2e):

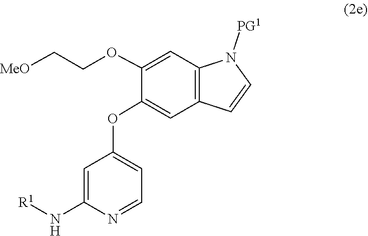

(where PG¹ and R¹ each represent the same groups as specified above),
2-d) step 2-d) in which PG¹ in compound (2e) obtained in step 2-c) is removed to produce compound (2f):

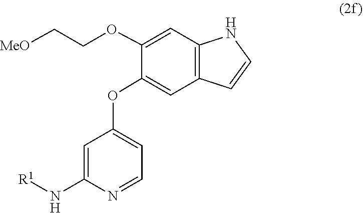

(where R¹ represents the same group as specified above),
2-e) step 2-e) in which compound (2f) obtained in step 2-d) and compound (2g-1) or compound (2g-2):

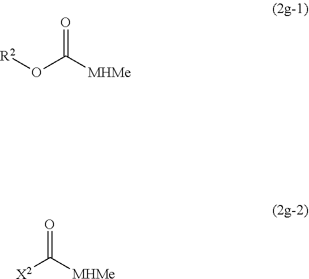

(where R² of formula (2g-1) is a C₁₋₆ alkyl group or C₆₋₁₀ aryl group, the C₁₋₆ alkyl group optionally having 1 to 3 identical or different substituents selected from the group consisting of halogen atoms and methoxy groups, and the C₆₋₁₀ aryl group optionally having 1 to 3 identical or different substituents selected from halogen atoms and methyl, methoxy and nitro groups, and X² of formula (2g-2) represents a halogen atom),
are reacted in the presence of a base to produce compound (2h):

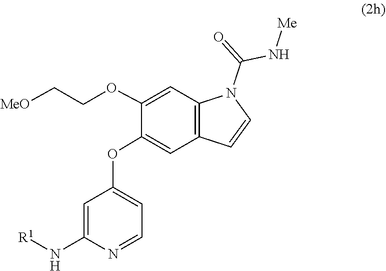

(where R¹ represents the same group as specified above), and
2-f) step 2-f) in which R¹ of compound (2h) obtained in step 2-e) is removed to produce compound (2i):

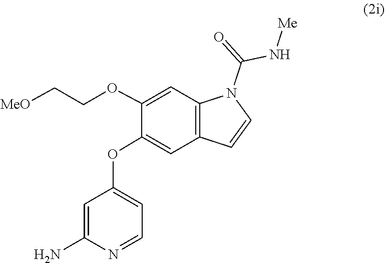 [SMILES: CNC(=O)n1ccc2cc(Oc3ccnc(N)c3)c(OCCOC)cc21]

and if necessary step 2-g) in which compound (2i) obtained in step 2-f) is converted to a salt,
[2] The production process according to [1], wherein compound (2b) is 4-nitropyridin-1-ium-oleate,
[3] The production process according to [1], wherein compound (2d) is 1,1,3,3-tetramethylbutylamine,
[4] The production process according to [1], wherein compound (2d) is cumylamine,
[5] The production process according to [1], wherein the activating agent is p-toluenesulfonyl chloride,
[6] A process for producing compound (1g):

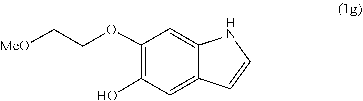 [SMILES: COCCOc1cc2[nH]ccc2cc1O]

wherein the process comprises:
1-a) step 1-a) in which 1,2-(methylenedioxy)-4-nitrobenzene and 4-bromobenzyl alcohol are reacted in the presence of a base to produce compound (1c):

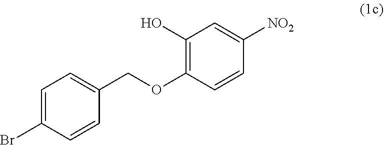 [SMILES: O=[N+]([O-])c1ccc(OCc2ccc(Br)cc2)c(O)c1]

1-b) step 1-b) in which compound (1c) obtained in step 1-a) and a methoxyethylating agent are reacted in the presence of a base to produce compound (1d):

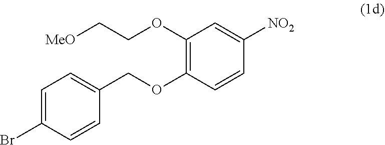 [SMILES: COCCOc1cc([N+](=O)[O-])ccc1OCc1ccc(Br)cc1]

1-c) step 1-c) in which compound (1d) obtained in step 1-b) and a cyanomethylating agent are reacted in the presence of a base to produce compound (1f):

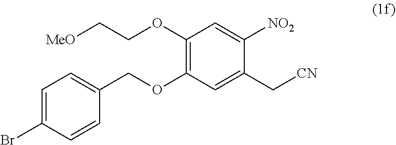 [SMILES: COCCOc1cc([N+](=O)[O-])c(CC#N)cc1OCc1ccc(Br)cc1]

and
1-d) step 1-d) in which compound (1g):

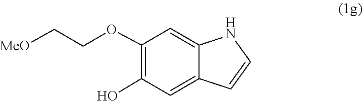 [SMILES: COCCOc1cc2[nH]ccc2cc1O]

is produced by conversion of the nitro group of compound (1f) obtained in step 1-c) to an amino group, removal of the 4-bromobenzyl group, and ring closure with an acid catalyst, and
[7] The production process according to [6], wherein a palladium catalyst and sulfuric acid are used in step 1-d).

### Advantageous Effects of Invention

The present invention can provide a production process allowing high-yield and efficient synthesis of key intermediates for production of E7090.

### DESCRIPTION OF EMBODIMENTS

The definitions of the symbols and terms used throughout the present specification will now be explained, prior to a more detailed description of the invention.

The term “C₁₋₆ alkyl group” as used herein means a straight-chain or branched substituent of 1 to 6 carbon atoms, which is a monovalent group derived by removing any one hydrogen atom from an aliphatic saturated hydrocarbon of 1 to 6 carbon atoms. Examples of C₁₋₆ alkyl groups include methyl, ethyl, 1-propyl, 2-propyl, 2-methyl-1-propyl, 2-methyl-2-propyl, 1-butyl, 2-butyl, 1-pentyl, 2-pentyl, 3-pentyl, 1-hexyl, 2-hexyl and 3-hexyl groups, with methyl and ethyl groups being preferred.

The term “C₆₋₁₀ aryl group” as used herein refers to an aromatic cyclic hydrocarbon group of 6 to 10 carbon atoms. Examples of C₆₋₁₀ aryl groups include phenyl, 1-naphthyl and 2-naphthyl groups, with phenyl being preferred.

The term “halogen atom” as used herein refers to fluorine, chlorine, bromine or iodine, and preferably chlorine or bromine.

The term “base” as used herein may refer to an inorganic base such as lithium hydroxide, sodium hydroxide, potassium hydroxide, lithium carbonate, sodium carbonate, potassium carbonate, potassium tert-butoxide, sodium tert-butoxide, sodium hydrogencarbonate, potassium hydrogencarbonate or cesium carbonate: an organometallic reagent such as butyllithium, methyllithium, lithium bis(trimethylsilyl)amide, sodium bis(trimethylsilyl)amide or potassium bis(trimethylsilyl)amide; a hydride such as lithium hydride, sodium hydride or potassium hydride: a heterocyclic compound such as imidazole, pyridine, dimethylpyridine, trimethylpyridine or 4-dimethylaminopyridine; or an organic amine such as triethylamine, N,N-diisopropylethylamine or diazabicycloundecene.

The term “compound” as used herein includes anhydrides, hydrates and solvates.

The term “salt” as used herein refers to, for example, inorganic acid salts (such as sulfates, nitrates, perchlorates, phosphates, carbonates, bicarbonates, hydrofluorides, hydrochlorides, hydrobromides and hydroiodides), organic carboxylic acid salts (such as acetates, oxalates, maleates, fumarates, succinates, tartrates and citrates), organic sulfonic acid salts (such as methanesulfonates, trifluoromethanesulfonates, ethanesulfonates, benzenesulfonates, toluenesulfonates and camphorsulfonates), and salts of acidic amino acids (such as aspartates and glutamates).

There are no particular restrictions on salts of compound (2i), and examples include inorganic acid salts, organic acid salts and acidic amino acid salts.

The production process of the invention will now be explained in greater detail.

### Production Process 1 Process for Producing Compound (1g)

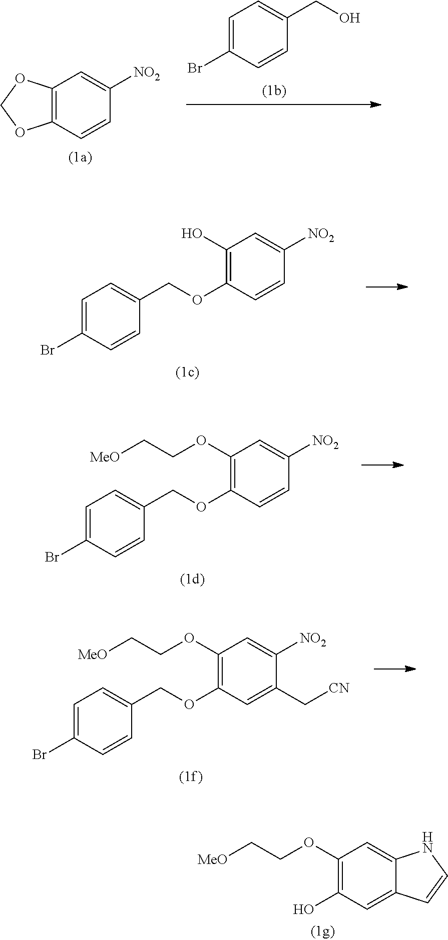 [SMILES: OCc1ccc(Br)cc1 ; O=[N+]([O-])c1ccc2c(c1)OCO2 ; O=[N+]([O-])c1ccc(OCc2ccc(Br)cc2)c(O)c1 ; COCCOc1cc([N+](=O)[O-])ccc1OCc1ccc(Br)cc1 ; COCCOc1cc([N+](=O)[O-])c(CC#N)cc1OCc1ccc(Br)cc1 ; COCCOc1cc2[nH]ccc2cc1O]

Step 1-a) is a step in which 1,2-(methylenedioxy)-4-nitrobenzene (compound (1a)) and 4-bromobenzyl alcohol (compound (1b)) are reacted in the presence of a base to obtain compound (1c).

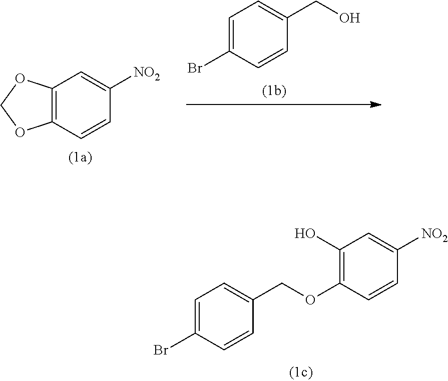 [SMILES: OCc1ccc(Br)cc1 ; O=[N+]([O-])c1ccc2c(c1)OCO2 ; O=[N+]([O-])c1ccc(OCc2ccc(Br)cc2)c(O)c1]

The 4-bromobenzyl alcohol may be used at 1.0 to 2.0 equivalents with respect to compound (1a). It is preferably used at 1.1 to 1.3 equivalents.

The base used may be potassium hydroxide, sodium hydroxide, potassium carbonate, sodium carbonate, cesium carbonate, potassium tert-butoxide, sodium tert-butoxide or sodium hydride. It is preferably sodium tert-butoxide. The base may be used at 1.0 to 5.0 equivalents with respect to compound (1a). It is preferably used at 1.0 to 3.0 equivalents.

The solvent is not particularly restricted so long as it dissolves the starting substance and does not interfere with the reaction, and for example, it may be N,N-dimethylformamide, dimethyl sulfoxide, N,N-dimethylacetamide, 1,3-dimethyl-2-imidazolidinone, tetrahydrofuran, or a mixed solvent thereof. It is preferably a mixed solvent of dimethyl sulfoxide and tetrahydrofuran.

The reaction temperature will generally differ depending on the starting substances, solvent and other reagents used in the reaction, but it is preferably 0° C. to 30° C. The temperature is preferably 5° C. to 20° C.

Step 1-b) is a step in which compound (1c) and a methoxyethylating agent are reacted in the presence of a base to obtain compound (1d).

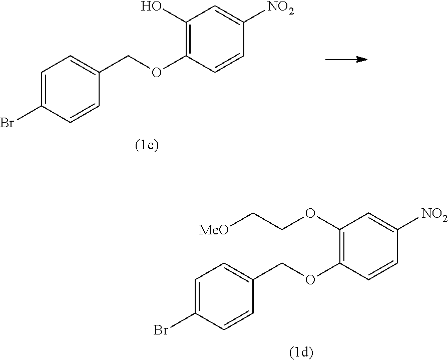 [SMILES: O=[N+]([O-])c1ccc(OCc2ccc(Br)cc2)c(O)c1 ; COCCOc1cc([N+](=O)[O-])ccc1OCc1ccc(Br)cc1]

Examples of methoxyethylating agents include 2-chloroethyl methyl ether, 2-bromoethyl methyl ether and 2-iodoethyl methyl ether. A preferred one is 2-bromoethyl methyl ether. The methoxyethylating agent may be used at 1.0 to 2.0 equivalents with respect to compound (1c). It is preferably used at 1.0 to 1.2 equivalents.

The base used may be potassium hydroxide, sodium hydroxide, potassium carbonate, sodium carbonate, cesium carbonate, potassium tert-butoxide, sodium tert-butoxide or sodium hydride. It is preferably potassium carbonate. The base may be used at 1.0 to 5.0 equivalents with respect to compound (1d). It is preferably used at 1.1 to 1.3 equivalents.

The solvent is not particularly restricted so long as it dissolves the starting substance and does not interfere with the reaction, and for example, it may be N,N-dimethylformamide, dimethyl sulfoxide, N,N-dimethylacetamide or 1,3-dimethyl-2-imidazolidinone. It is preferably N,N-dimethylformamide.

The reaction temperature will generally differ depending on the starting substances, solvent and other reagents used in the reaction, but it is preferably room temperature to 80° C. The temperature is preferably 40° C. to 70° C.

Step 1-c) is a step in which compound (1d) and a cyanomethylating agent are reacted in the presence of a base to obtain compound (1f).

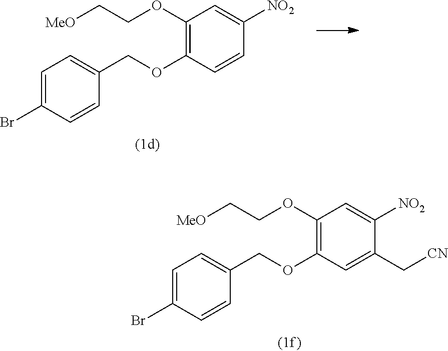 [SMILES: COCCOc1cc([N+](=O)[O-])ccc1OCc1ccc(Br)cc1 ; COCCOc1cc([N+](=O)[O-])c(CC#N)cc1OCc1ccc(Br)cc1]

Examples of cyanomethylating agents include 4-chlorophenoxyacetonitrile, 4-bromophenoxyacetonitrile, phenoxyacetonitrile, 2-chloroacetonitrile, 2-bromoacetonitrile, 2-iodoacetonitrile and (cyanomethyl)trimethylammonium iodide. A preferred one is 4-chlorophenoxyacetonitrile. The cyanomethylating agent may be used at 1.0 to 2.0 equivalents with respect to compound (1d). It is preferably used at 1.2 to 1.4 equivalents.

The base used may be potassium hydroxide, sodium hydroxide, potassium carbonate, sodium carbonate, cesium carbonate, potassium tert-butoxide, sodium tert-butoxide or sodium hydride. It is preferably potassium tert-butoxide. The base may be used at 1.0 to 5.0 equivalents with respect to compound (1d). It is preferably used at 2.0 to 4.0 equivalents.

The solvent is not particularly restricted so long as it dissolves the starting substance and does not interfere with the reaction, and for example, it may be N,N-dimethylformamide, dimethyl sulfoxide, N,N-dimethylacetamide or 1,3-dimethyl-2-imidazolidinone. It is preferably N,N-dimethylformamide.

The reaction temperature will generally differ depending on the starting substances, solvent and other reagents used in the reaction, but it is preferably −70° C. to room temperature. The temperature is preferably −70° C. to −50° C.

Step 1-d) is a step in which compound (1g) is obtained by conversion of the nitro group in compound (1f) to an amino group, removal of the 4-bromobenzyl group, and ring closure with an acid catalyst.

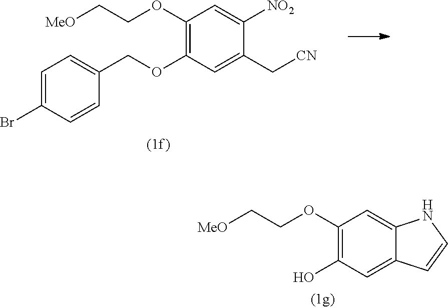 [SMILES: COCCOc1cc([N+](=O)[O-])c(CC#N)cc1OCc1ccc(Br)cc1 ; COCCOc1cc2[nH]ccc2cc1O]

A reduction catalyst may be used in a hydrogen atmosphere for conversion of the nitro group to an amino group and removal of the 4-bromobenzyl group. Examples of reduction catalysts include palladium carbon, palladium black and platinum oxide. It is preferred to use palladium carbon under a hydrogen atmosphere.

The reaction solvent is not particularly restricted so long as it dissolves the starting substance and does not interfere with the reaction, and for example, it may be tetrahydrofuran, methanol, ethanol, water, or a mixture thereof such as tetrahydrofuran and water, tetrahydrofuran and methanol or tetrahydrofuran and ethanol. It is preferably a mixed solvent of tetrahydrofuran and water.

The acid catalyst may be hydrochloric acid, sulfuric acid or acetic acid. It is preferably sulfuric acid. The concentration of the acid catalyst that is used may be 0.01 N to 1.0 N. The concentration is preferably 0.01 N to 0.2 N. The reaction temperature will generally differ depending on the starting substances, solvent and other reagents used in the reaction, but it is preferably room temperature to 60° C. The temperature is preferably 30° C. to 50° C.

### Production Process 2 Process for Producing Compound (2i)

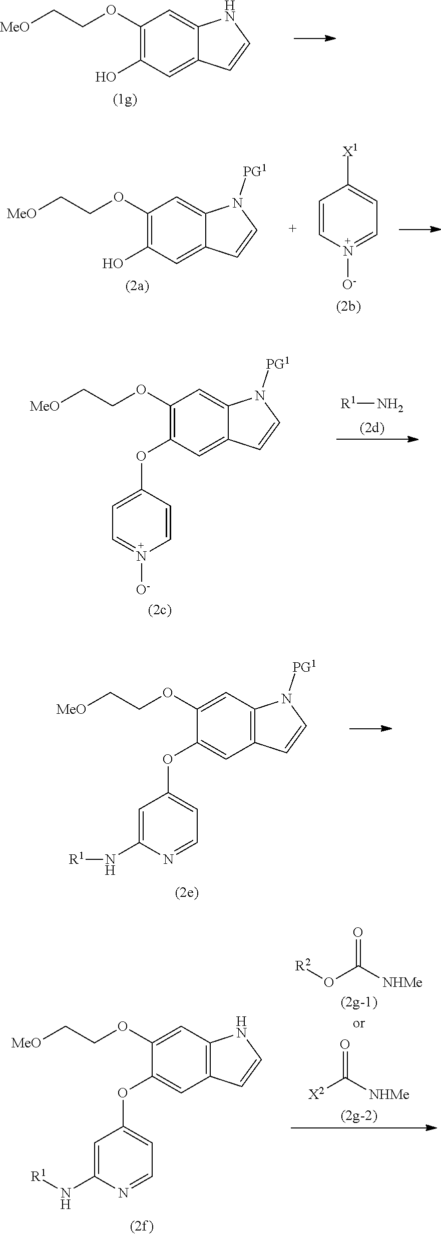

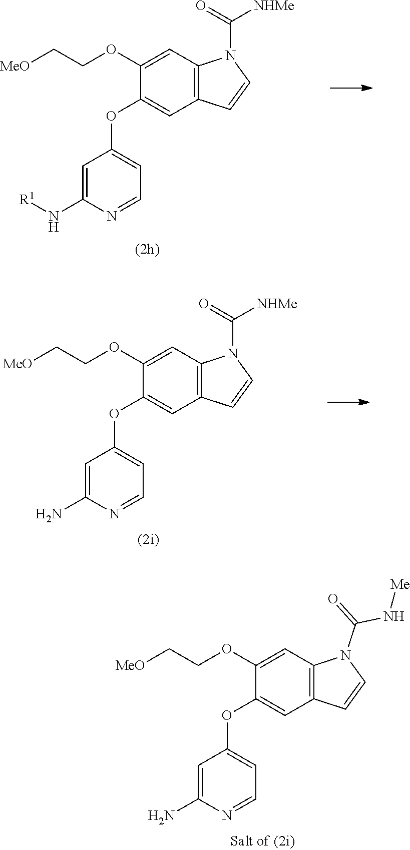

Step 2-a) is a step of introducing a protecting group into compound (1g) to obtain compound (2a).

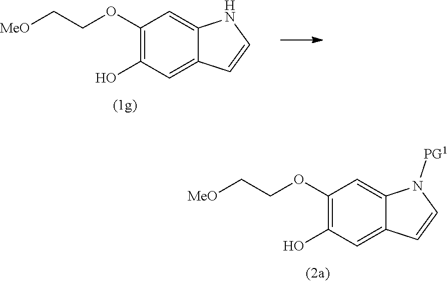

The protecting group introduced may be a tert-butoxycarbonyl, benzyloxycarbonyl, benzoyl, acetyl or trifluoroacetyl group. It is preferably a tert-butoxycarbonyl group. A di-tert-butyl-dicarbonate or tert-butoxycarbonyl chloride compound may be used to introduce the tert-butoxycarbonyl group. Such a compound is preferably di-tert-butyl-dicarbonate. The di-tert-butyl-dicarbonate may be used at 1 to 5 equivalents with respect to compound (1g). It is preferably used at 2.0 to 2.5 equivalents.

When di-tert-butyl-dicarbonate is used, the base used is preferably triethylamine, N-methylimidazole or N,N-dimethylaminopyridine (DMAP). Preferably, N,N-dimethylaminopyridine (DMAP) is used. When di-tert-butyl-dicarbonate is used, the N,N-dimethylaminopyridine (DMAP) may be used at 0.01 to 2.0 equivalents with respect to compound (1g). It is preferably used at 0.05 to 0.2 equivalents.

The solvent is not particularly restricted so long as it dissolves the starting substance and does not interfere with the reaction, and for example, it may be tetrahydrofuran, N,N-dimethylformamide, acetonitrile or ethyl acetate. The solvent is preferably tetrahydrofuran.

The reaction temperature will generally differ depending on the starting substances, solvent and other reagents used in the reaction, but it is preferably 0° C. to 60° C. The temperature is preferably 20° C. to 30° C.

In step 2-a), the hydroxyl groups may be selectively deprotected, under suitable conditions depending on the protecting group. For example, deprotection can be easily carried out under hydrolysis conditions for tert-butoxycarbonyl or benzyloxycarbonyl groups. For hydrolysis of a di-tert-butoxycarbonyl compound, in particular, the base used may be sodium hydroxide, potassium hydroxide, sodium carbonate or potassium carbonate, for example. The base is preferably potassium carbonate. The potassium carbonate may be used at 0.7 to 1.2 equivalents with respect to compound (1g). It is preferably used at 0.7 to 0.9 equivalents.

The solvent is not particularly restricted so long as it dissolves the starting substance and does not interfere with the reaction, and for example, it may be methanol, ethanol, isopropyl alcohol, acetonitrile, water, or a mixture of these solvents. The solvent is preferably methanol.

The reaction temperature will generally differ depending on the starting substances, solvent and other reagents used in the reaction, but it is preferably 25° C. to 50° C. The temperature is preferably 30° C. to 40° C.

Step 2-b) is a step in which compound (2a) and compound (2b) are reacted in the presence of a base to obtain compound (2c).

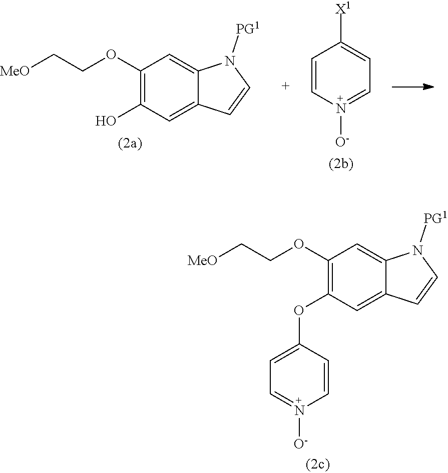

Compound (2b) may be 4-chloropyridin-1-ium-oleate, 4-bromopyridin-1-ium-oleate or 4-nitropyridin-1-ium-oleate. It is preferably 4-nitropyridin-1-ium-oleate. Compound (2b) may be used at 1.0 to 1.4 equivalents with respect to compound (2a). It is preferably used at 1.1 to 1.3 equivalents.

The base used may be potassium carbonate, cesium carbonate or potassium tert-butoxide, or a 48% potassium hydroxide aqueous solution. It is preferably cesium carbonate. The base may be used at 1 to 3 equivalents with respect to compound (2a). It is preferably used at 1.4 to 1.6 equivalents.

The solvent is not particularly restricted so long as it dissolves the starting substance and does not interfere with the reaction, and for example, it may be N,N-dimethylformamide, N,N-dimethylacetamide, or dimethyl sulfoxide 1,3-dimethyl-2-imidazolidinone. It is preferably dimethyl sulfoxide.

The reaction temperature will generally differ depending on the starting substances, solvent and other reagents used in the reaction, but it is preferably 0° C. to 60° C. The temperature is preferably 30° C. to 50° C.

Step 2-c) is a step in which compound (2c) and compound (2d) are reacted in the presence of an activating agent to obtain compound (2e).

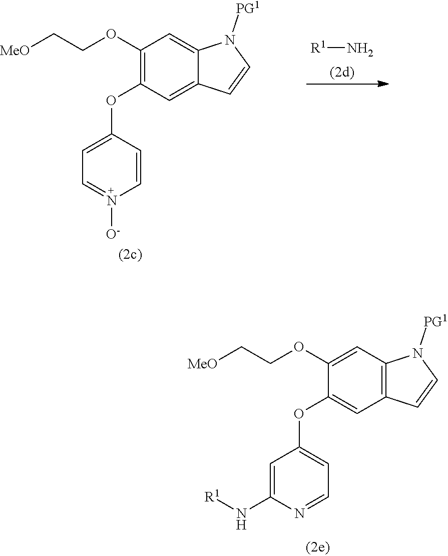

Examples for compound (2d) include tert-butylamine, tert-pentylamine, cumylamine and tert-octylamine. It is preferably tert-octylamine. Compound (2d) may be used at 1 to 15 equivalents with respect to compound (2c). It is preferably used at 5 to 10 equivalents.

The activating agent may be p-toluenesulfonyl chloride, benzenesulfonyl chloride, p-chlorosulfonyl chloride, p-methoxysulfonyl chloride, 2-methylsulfonyl chloride, 1-naphthylsulfonyl chloride, 2,4,6-trimethylsulfonyl chloride or 2,4,6-triphenylsulfonyl chloride. It is preferably p-toluenesulfonyl chloride. The equivalent amount of the activating agent may be 1 to 5 equivalents with respect to compound (2c). It is preferably used at 1.5 to 2.5 equivalents.

The solvent is not particularly restricted so long as it dissolves the starting substance and does not interfere with the reaction, and for example, it may be trifluoromethylbenzene, toluene, ethyl acetate, isopropyl acetate, tetrahydrofuran, 2-methyltetrahydrofuran, tert-butyl methyl ether, dimethoxyethane, cyclopentyl methyl ether, 4-methyltetrahydropyran, water, or a mixture of the foregoing. It is preferably a mixed solvent of toluene, 4-methyltetrahydropyran and water.

The reaction temperature will generally differ depending on the starting substances, solvent and other reagents used in the reaction, but it is preferably −50° C. to room temperature, and preferably −20° C. to 10° C.

Step 2-d) is a step in which PG¹ is removed from compound (2e) to obtain compound (2f).

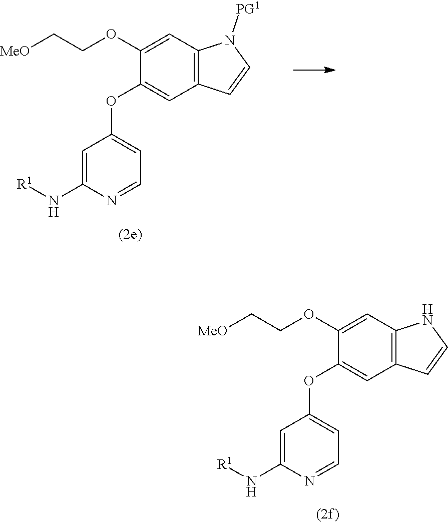

In step 2-d), deprotection may be carried out under deprotection conditions suited for the protecting group. In the case of a tert-butoxycarbonyl or benzyloxycarbonyl group, for example, deprotection may be carried out under basic conditions. The base used may be sodium hydroxide, potassium hydroxide, sodium tert-butoxide, potassium tert-butoxide, potassium carbonate, tert-butylamine, sodium methoxide or sodium ethoxide, for example. It is preferably sodium hydroxide. The base is used at preferably 1 to 10 equivalents and more preferably 2 to 4 equivalents with respect to compound (2e).

The solvent is not particularly restricted so long as it dissolves the starting substance and does not interfere with the reaction, and for example, it may be ethanol, tetrahydrofuran, dimethyl sulfoxide, methanol, water, or a mixture of these solvents. In the case of a tert-butoxycarbonyl group, for example, the solvent is preferably a mixture of ethanol, tetrahydrofuran and water. The reaction temperature will generally differ depending on the starting substances, solvent and other reagents used in the reaction, but it is preferably 30° C. to 70° C. The temperature is preferably 40° C. to 60° C.

Step 2-e) is a step in which compound (2f) and compound (2g-1) or compound (2g-2) are reacted in the presence of a base to obtain compound (2h).

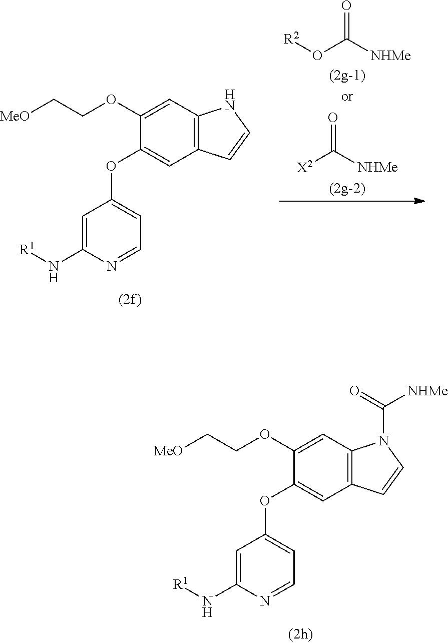

Compound (2g-1) or compound (2g-2) may be phenylmethyl carbamate, ethylmethyl carbamate, methylaminocarbonyl chloride or methylaminocarbonyl bromide, for example. A preferred compound is phenylmethyl carbamate. Phenylmethyl carbamate, for example, may be used at 1.0 to 2.0 equivalents with respect to compound (2f). It is preferably used at 1.2 to 1.4 equivalents.

The base used may be sodium hydroxide, potassium hydroxide, sodium tert-butoxide or potassium tert-butoxide. It is preferably potassium tert-butoxide. The base may be used at 0.1 to 3.0 equivalents with respect to compound (2f). It is preferably used at 1.0 to 1.5 equivalents.

The solvent is not particularly restricted so long as it dissolves the starting substance and does not interfere with the reaction, and for example, it may be tetrahydrofuran, dimethylformamide or dimethyl sulfoxide, or a mixture of these solvents. It is preferably a mixed solvent of tetrahydrofuran and dimethyl sulfoxide.

The reaction temperature will generally differ depending on the starting substances, solvent and other reagents used in the reaction, but it is preferably −10° C. to room temperature. The temperature is more preferably 0° C.

Step 2-f) is a step in which R¹ is removed from compound (2h) to obtain compound (2i), and then if necessary a salt of compound (2i) is obtained.

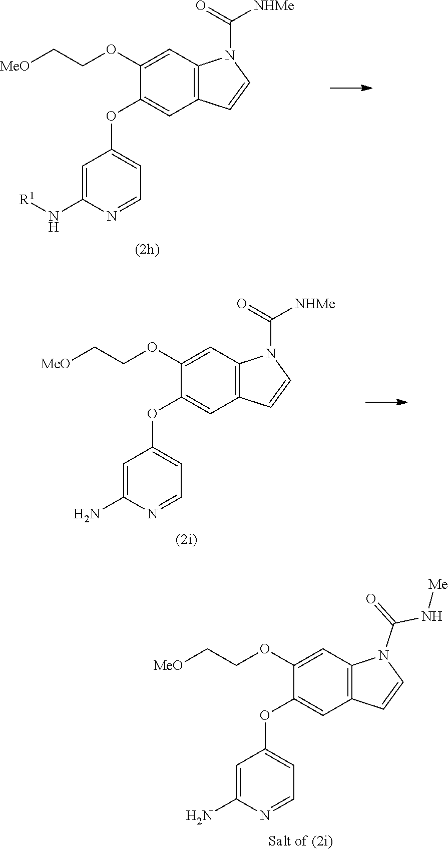

The conditions for removal of R¹ in step 2-f) may be selected depending on the type of R¹ group. When R¹ is a cumyl or octyl group, for example, the acid used may be hydrochloric acid, sulfuric acid, formic acid or methanesulfonic acid. It is preferably methanesulfonic acid.

Compound (2i) can be converted to a salt by acid treatment. The salt may be, for example, inorganic acid salts (such as sulfates, nitrates, perchlorates, phosphates, carbonates, bicarbonates, hydrofluorides, hydrochlorides, hydrobromides and hydroiodides), organic carboxylic acid salts (such as acetates, oxalates, maleates, tartrates, fumarates and citrates), organic sulfonic acid salts (such as methanesulfonates, trifluoromethanesulfonates, ethanesulfonates, benzenesulfonates, toluenesulfonates and camphorsulfonates), and salts of amino acids (such as aspartates and glutamates). The salt is preferably a methanesulfonate. Compound (2i) can be stably stored for long periods if it is converted to a salt. A process for synthesizing a methanesulfonate of compound (2i) by removal of R¹ from compound (2h) will now be explained as an example.

Methanesulfonic acid may be used at 1 to 20 equivalents with respect to compound (2h). It is preferably used at 5 to 10 equivalents.

The reaction solvent is not particularly restricted so long as it dissolves the starting substance and does not interfere with the reaction, and for example, it may be ethanol, methanol or dimethoxyethane, or a mixture of these solvents. It is preferably a mixed solvent of ethanol and methanol. The reaction temperature will generally differ depending on the starting substances, solvent and other reagents used in the reaction, but it is preferably room temperature to 80° C. The temperature is preferably 20° C. to 50° C.

### EXAMPLES

The present invention will now be explained in greater detail by Examples. However, the invention is not limited to these Examples. The abbreviations used here throughout are those commonly known to those skilled in the art, and a few are indicated below:

The ¹H-NMR spectra were measured using a BRUCKER AVANCE NEO 400 (400 MHZ), BRUCKER AVANCE III 500 (500 MHz), BRUCKER AVANCE 600 (600 MHz) or BRUCKER AVANCE NEO 700 (700 MHz).

The chemical shifts in the proton nuclear magnetic resonance (¹H-NMR) spectra are recorded in δ units (ppm) with respect to tetramethylsilane, and the coupling constants are recorded in Hertz (Hz). The patterns are indicated as s: singlet, d: doublet, br. broad or m: multiplet.

The term “room temperature” in the Examples generally refers to a temperature between about 10° C. and 35° C. The percentage values are weight percentages, unless otherwise specified.

### Example 1: Production of 6-(2-methoxyethoxy)-1H-indole-5-ol (compound (1g))

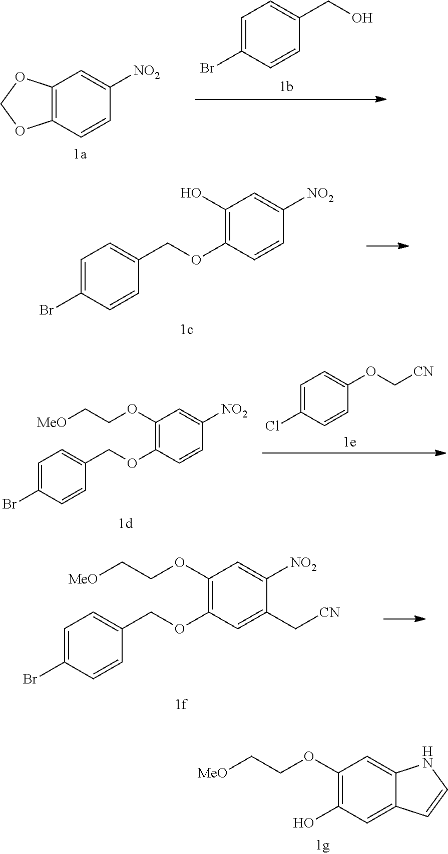

### Production Example 1: Production of 2-[(4-bromobenzyl)oxy]-5-nitrophenol (1c)

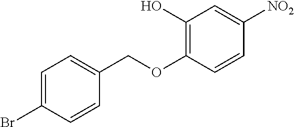 [SMILES: O=[N+]([O-])c1ccc(OCc2ccc(Br)cc2)c(O)c1]

A solution of 4-bromobenzyl alcohol (120.6 kg, 645 mol, 1.2 eq.) and sodium tert-butoxide (103.3 kg, 1075 mol, 2.0 eq.) in dimethyl sulfoxide (450 L) and tetrahydrofuran (157 L) was prepared at 13° C. To this solution was added dropwise a solution of 1,2-(methylenedioxy)-4-nitrobenzene (89.8 kg, 537 mol) in tetrahydrofuran (726 L) and dimethyl sulfoxide (269 L) at 10 to 15° C., and then tetrahydrofuran (14 L) was added and the mixture was stirred at 14° C. for 2 hours. A mixture of water (500 L) and 35% hydrochloric acid (83 L) was added dropwise to the reaction mixture at 30° C. or lower. After then adding isopropyl acetate (450 L), the organic layer was separated off. The organic layer was rinsed with a 7% aqueous sodium bicarbonate solution (290 kg), and then rinsed with 9% brine (296 kg). Isopropyl acetate (900 L) was added to the organic layer, and the mixture was concentrated under reduced pressure to 547 L. Isopropyl acetate (900 L) was added to the concentrated residue, and the mixture was concentrated under reduced pressure to 547 L. Hexane (450 L) was added to the concentrated residue at 26 to 32° C., and after cooling to 10° C. or lower, the precipitated solid was filtered. The crystals were rinsed with an isopropyl acetate (90 L)/methanol (27 L/hexane (180 L) mixture, and the resulting crystals were dried under reduced pressure at an internal temperature of 50° C. or lower to obtain 125 kg of the title compound.

¹H NMR Spectrum (DMSO-d₆) δ (ppm): 5.23 (2H, s), 7.17 (1H, d, J-9.1 Hz), 7.44 (2H, br d, J=8.3 Hz), 7.60 (2H, br d, J=8.3 Hz), 7.63 (1H, d, J=2.6 Hz), 7.71 (1H, dd, J=9.1, 2.6 Hz)

### Production Example 2-1 Production of 1-[(4-bromobenzyl)oxy]-2-(2-methoxyethoxy)-4-nitrobenzene (1d) (1)

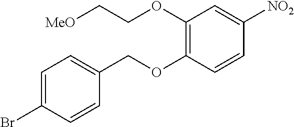 [SMILES: COCCOc1cc([N+](=O)[O-])ccc1OCc1ccc(Br)cc1]

Potassium carbonate (180 g, 1301 mmol, 1.2 eq.) was added to a solution of 2-[(4-bromobenzyl)oxy]-5-nitrophenol (351.4 g, 1084 mmol) in dimethylformamide (1750 mL), and the mixture was stirred. Next, 2-bromoethyl methyl ether (166 g, 1193 mol, 1.1 eq.) was added and the mixture was stirred for 6 days. Ethyl acetate (9000 mL) and water (3000 mL) were added prior to liquid separation of the mixture. After rinsing the organic layer 4 times with water (350 mL), the organic layer was concentrated under reduced pressure at 50° C. Heptane (1500 mL) was added to the concentrated residue to form a suspension, which was filtered to obtain crystals. The filtrate was concentrated under reduced pressure at 50° C., and heptane (1000 mL) was added to the concentrated residue to form a suspension, which was filtered to obtain crystals. The obtained crystals were combined and dried under reduced pressure at 50° C. to obtain 408 g of the title compound.

¹H NMR Spectrum (DMSO-d₆) δ (ppm): 3.28 (3H, s), 3.68 (2H, t, J=4.5 Hz), 4.23 (2H, t, J-4.2 Hz), 5.26 (2H, s), 7.23 (1H, d, J=9.1 Hz), 7.41 (2H, br d, J=7.9 Hz), 7.60 (2H, br d, J=7.9 Hz), 7.79 (1H, d, J-2.3 Hz), 7.88 (1H, dd, J=8.7, 1.9 Hz)

### Production Example 2-2: Production of 1-[(4-bromobenzyl)oxy]-2-(2-methoxyethoxy)-4-nitrobenzene (1d) (2)

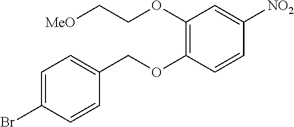 [SMILES: COCCOc1cc([N+](=O)[O-])ccc1OCc1ccc(Br)cc1]

To a solution of 2-bromoethyl methyl ether (29.5 kg, 212 mol, 1.1 eq.) in dimethylformamide (294 kg) were added 2-[(4-bromobenzyl)oxy]-5-nitrophenol (62.5 kg, 193 mol) and potassium carbonate (32.0 kg, 232 mol, 1.2 eq.), and the mixture was stirred at 56 to 60° C. for 6 hours. After cooling, ethyl acetate (1252 L) and water (375 L) were added. After liquid separation, the organic layer was rinsed with water (188 L), and ethyl acetate (83 L) and a 5% aqueous sodium chloride solution (198 kg) were added to the organic layer prior to liquid separation. The organic layer was concentrated under reduced pressure to 737 L, and then a suspension of 1-[(4-bromobenzyl)oxy]-2-(2-methoxyethoxy)-4-nitrobenzene (22.2 g) in methanol (1.7 kg) was added. Methanol (738 L) was added dropwise at 0 to −4° C., and the precipitated crystals were filtered out. The crystals were rinsed with methanol (189 L) and the resulting crystals were dried under reduced pressure at an internal temperature of 50° C. or lower to obtain 66.0 kg of the title compound.

### Production Example 3: Production of {5-[(4-bromobenzyl)oxy]-4-(2-methoxyethoxy)-2-nitrophenyl}acetonitrile (1f)

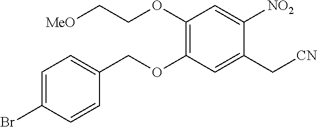 [SMILES: COCCOc1cc([N+](=O)[O-])c(CC#N)cc1OCc1ccc(Br)cc1]

To a solution of potassium tert-butoxide (14.7 kg, 131 mol, 3 eq.) in dimethylformamide (168 L) was added dropwise a solution of 1-[(4-bromobenzyl)oxy]-2-(2-methoxyethoxy)-4-nitrobenzene (16.7 kg, 43.7 mol) and 4-chlorophenoxyacetonitrile (9.5 kg, 56.7 mol, 1.3 eq.) in dimethylformamide (80 L) at −60 to −55° C., and after adding dimethylformamide (21 L), the mixture was stirred at −63 to −58° C. for 2 hours. A mixture of ethyl acetate (351 L) and acetic acid (8 L) was added dropwise to the reaction mixture. After adding a 5% aqueous sodium chloride solution (168 kg), the organic layer was rinsed twice with a 5% aqueous sodium chloride solution (84 kg, 85 kg). Ethyl acetate (34 L) was added to the organic layer, and then rinsed with a 5% aqueous sodium chloride solution (85 kg). The organic layer was concentrated under reduced pressure, and tert-butyl methyl ether (134 L) and methanol (13 L) were added to the concentrated residue, after which the mixture was cooled to 0 to 2° C. and the precipitated solid was filtered out. The obtained crystals were rinsed with a tert-butylmethyl ether (30 L)/ethyl acetate (3 L)/methanol (3 L) mixture, and the resulting crystals were dried under reduced pressure at an internal temperature of 50° C. or lower to obtain 10.7 kg of the title compound. ¹H-NMR Spectrum (CDCl₃) δ (ppm): 3.46 (3H, s), 3.81 (2H, t, J=4.8 Hz), 4.19 (2H, s), 4.25 (2H, t. J=4.4 Hz), 5.23 (2H, s), 7.14 (1H, s), 7.35 (2H, d, J=8.0 Hz), 7.54 (2H, d, J-8.4 Hz), 7.84 (1H, s)

### Production Example 4: Production of 6-(2-methoxyethoxy)-1H-indol-5-ol (1g)

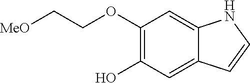 [SMILES: COCCOc1cc2[nH]ccc2cc1O]

To a solution of {5-[(4-bromobenzyl)oxy]-4-(2-methoxyethoxy)-2-nitrophenyl}acetonitrile (28.2 kg, 66.9 mol) in tetrahydrofuran (282 L) were added 10% palladium/carbon (5.7 kg), water (28.2 L) and 98% purified concentrated sulfuric acid (0.21 kg), and the mixture was stirred for 5 hours at 40 to 45° C. and a hydrogen pressure of 0.02 to 0.15 MPa. After the reaction, the catalyst was filtered out, the catalyst residue was rinsed with ethyl acetate (284 L), and the resulting filtrate was separated. A mixture of water (129 L)/hydrochloric acid (12.8 kg) was added dropwise to the organic layer prior to liquid separation. After rinsing the organic layer with 5% sodium bicarbonate solution (141 kg) and 3% brine (146 kg), the organic layer was concentrated under reduced pressure to 85 L. Ethyl acetate (144 L) and a 10% aqueous sodium chloride solution (57 kg) were added to the concentrated residue, and the organic layer was rinsed. The organic layer was concentrated under reduced pressure to 85 L. Heptane (28 L) and sodium sulfate (14.1 kg) were added to the obtained concentrated residue and the mixture was stirred. The liquid mixture was passed through NH silica gel (28.2 kg) that had been wetted with ethyl acetate (141 L), for purification. The NH silica gel was then rinsed off with an ethyl acetate (226 L)heptane (57 L) mixture, and combined with the purified mixture. After concentration under reduced pressure to 85 L at 50° C., heptane (64 L) was added dropwise to the concentrated residue, and the mixture was concentrated under reduced pressure to 85 L at 50° C. Ethyl acetate (27 L) and heptane (60 L) were added and the mixture was cooled to 0 to 10° C., after which the precipitated solid was filtered off. The obtained crystals were rinsed with a heptane (45 L)/ethyl acetate (14 L) mixture. The obtained crystals were dried under reduced pressure at an internal temperature of 50° C. or lower to obtain 7.4 kg of the title compound.

¹H NMR Spectrum (DMSO-d₆) δ (ppm): 3.32 (3H, s), 3.66-3.69 (2H, m), 4.04-4.07 (2H, m), 6.16 (1H, t, J=2.1 Hz), 6.88 (2H, d, J=4.2 Hz), 7.07 (1H, dd, J=2.8, 2.5 Hz), 8.08 (1H, s), 10.57 (1H, br s)

### Production Example 4-2: Recrystallization of 6-(2-methoxyethoxy)-1H-indol-5-ol

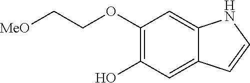 [SMILES: COCCOc1cc2[nH]ccc2cc1O]

A mixture of 6-(2-methoxyethoxy)-1H-indole-5-ol (21.2 kg, 102.3 mol) and ethyl acetate (97 L) was stirred while heating at an internal temperature of 50 to 60° C., and dissolution was confirmed. The solution was clarified by filtration and washed in with ethyl acetate (10 L). It was then cooled to an internal temperature of 40 to 45° C., and after confirming precipitation of crystals, it was stirred for 1 hour at the same temperature. The suspension was cooled to an internal temperature of −10 to 0° C. over a period of 6 hours, and then stirred for 14 hours. After then adding n-heptane (145 kg) dropwise over a period of 1.5 hours, the mixture was stirred for 3 hours at an internal temperature of −10 to 0° C. The suspension was filtered and rinsed with a mixture of ethyl acetate (5.7 kg) and n-heptane (8.7 kg). The obtained crystals were dried under reduced pressure at 40° C. to obtain 20.2 kg of the title compound.

### Example 2: Production of 5-((2-aminopyridin-4-yl)oxy)-6-(2-methoxyethoxy)-N-methyl-1H-indole-1-carboxamide methanesulfonate (methanesulfonate of compound (2i)

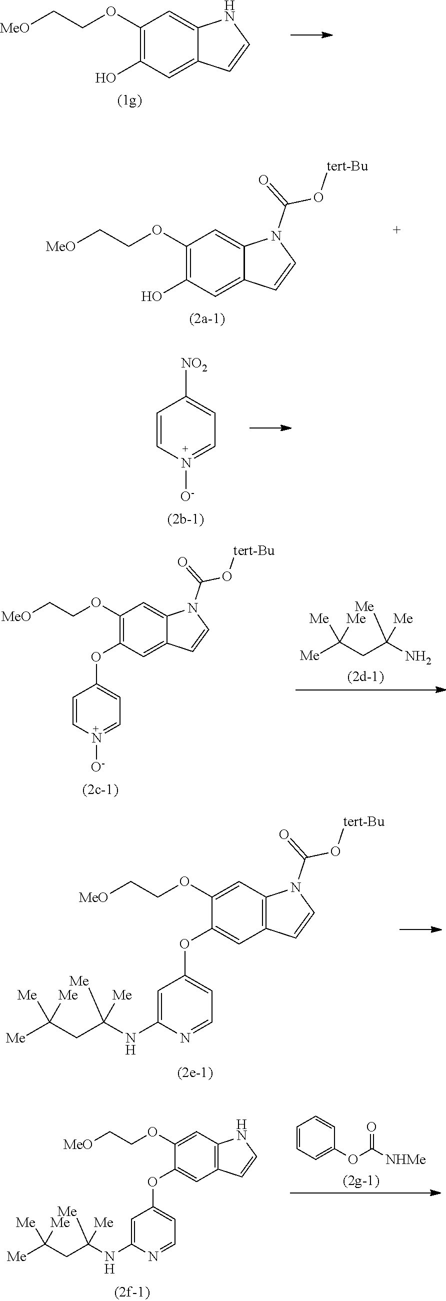

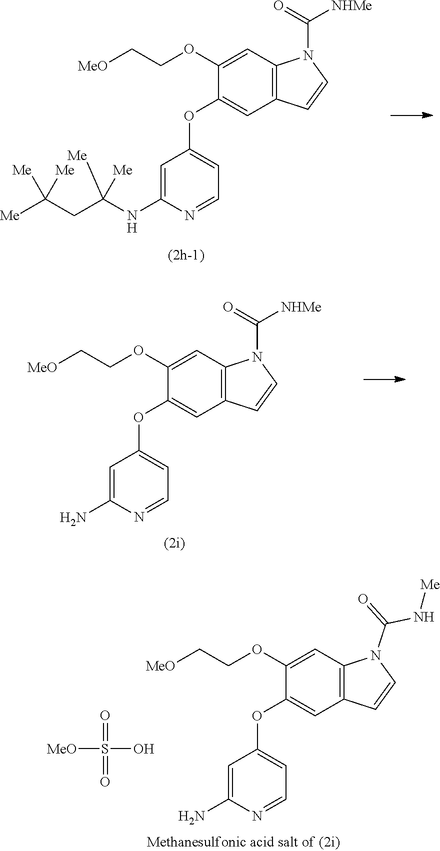 [SMILES: CNC(=O)n1ccc2cc(Oc3ccnc(NC(C)(C)CC(C)(C)C)c3)c(OCCOC)cc21 ; CNC(=O)n1ccc2cc(Oc3ccnc(N)c3)c(OCCOC)cc21 ; COS(=O)(=O)O ; CNC(=O)n1ccc2cc(Oc3ccnc(N)c3)c(OCCOC)cc21]

### Production Example 5: Production of tert-butyl-5-hydroxy-6-(2-methoxyethoxy)-1H-indole-1-carboxylate (2a-1)

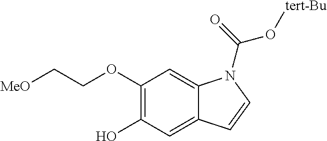

To a suspension of 6-(2-methoxyethoxy)-1H-indole-5-ol (19.8 kg, 95.6 mol) and 4-(dimethylamino)pyridine (1.17 kg, 9.55 mol, 0.1 eq.) in tetrahydrofuran (70.4 kg) was added dropwise a solution of di-tert-butyl-dicarbonate (45.9 kg, 210 mol, 2.2 eq.) in tetrahydrofuran (26.4 kg) under a nitrogen atmosphere, at 25° C. or lower, and it was washed into the mixture with tetrahydrofuran (8.8 kg) and the resulting mixture was stirred for 25° C. for 1 hour. Upon completion of the reaction, the reaction mixture was concentrated under reduced pressure to 90 L, at 40° C. or lower. Methanol (78.3 kg) and potassium carbonate (10.6 kg, 76.4 mol, 0.8 eq.) were added to the obtained concentrate under a nitrogen atmosphere, and the mixture was stirred for 14 hours at 34° C. After cooling the reaction mixture to 25° C., ethyl acetate (178.6 kg) and water (138.6 kg) were added, and 5 N hydrochloric acid (15.2 kg of 35% hydrochloric acid, 16.3 kg water) was added dropwise. After liquid separation, the organic layer was rinsed with 5% brine (3.0 kg salt, 56.4 kg water). The organic layer was concentrated under reduced pressure to 100 L at 50° C. or lower, and then toluene (85.6 kg) was used twice for azeotropic distillation. Dimethyl sulfoxide (87.1 kg) was added to the obtained concentrate and the mixture was concentrated under reduced pressure to 100 L, to obtain a crude product of the title compound (29.4 kg content as 100%) as a dimethyl sulfoxide solution (100 L).

¹H NMR Spectrum (DMSO-d₆) δ (ppm): 1.61 (9H, s), 3.32 (3H, s), 3.68-3.71 (2H, m), 4.08-4.11 (2H, m), 6.49 (1H, d, J=3.6 Hz), 6.95 (1H, s), 7.44 (1H, d, J=3.6 Hz), 7.59 (1H, s), 8.75 (1H, s)

### Production Example 6: Production of tert-butyl-6-(2-methoxyethoxy)-5-[(1-oxo-1λ⁵-pyridin-4-yl)oxy]-1H-indole-1-carboxylate (2c-1)

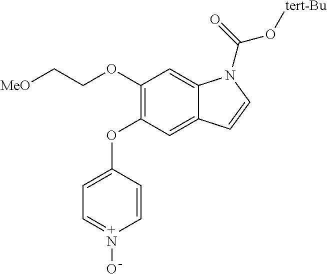

To a solution of tert-butyl-5-hydroxy-6-(2-methoxyethoxy)-1H-indole-1-carboxylate in dimethyl sulfoxide (100 L) (content: 29.4 kg, 95.6 mol) were added 4-nitropyridin-1-ium-oleate (16.1 kg, 115 mol, 1.2 eq.) and dimethyl sulfoxide (106.5 kg). Cesium carbonate (46.7 kg, 143 mol, 1.5 eq.) was added in 7 portions every 30 minutes at 40° C. under a nitrogen atmosphere, and the mixture was then stirred for 3 hours. Upon completion of the reaction, the reaction mixture was cooled to 15° C. or lower, 2-methyltetrahydrofuran (252.5 kg) was added, and water (293.6 kg) was added dropwise prior to liquid separation. The aqueous layer was extracted with 2-methyltetrahydrofuran (126.3 kg), and the combined organic layer was rinsed with 10% brine (8.8 kg salt, 79.2 kg water). The obtained organic layer was concentrated under reduced pressure to 130 L at 50° C. or lower, and further subjected to azeotropic distillation 3 times with toluene (127.0 kg). Toluene (48.4 kg) was added to the concentrate, the mixture was heated to 55° C., and the resulting suspension was cooled to −15° C., after which it was filtered and rinsed with toluene (51.0 kg). The obtained crystals were dried under reduced pressure at 50° C. or lower to obtain 28.61 kg of the title compound.

¹H NMR Spectrum (DMSO-d₆) δ (ppm): 1.63 (9H, s), 3.16 (3H, s), 3.49-3.52 (2H, m), 4.09-4.12 (2H, m), 6.65 (1H, d, J=3.8 Hz), 6.83-6.87 (2H, m), 7.48 (1H, s), 7.62 (1H, d, J=3.6 Hz), 7.82 (1H, br s), 8.04-8.07 (2H, m)

### Production Example 7: Production of 6-(2-methoxyethoxy)-N-methyl-5-({2-[(2,4,4-trimethylpentan-2-yl)amino]pyridin-4-yl}oxy)-1H-indole-1-carboxamide (2e-1)

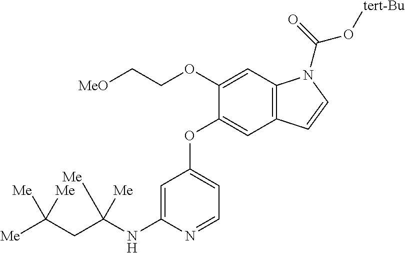

After adding 1,1,3,3-tetramethylbutylamine (64.4 kg, 498 mol, 7.0 eq.) and water (0.4 kg, 0.3 eq.) to a suspension of tert-butyl-6-(2-methoxyethoxy)-5-[(1-oxo-1λ-pyridin-4-yl)oxy]-1H-indole-1-carboxy late (28.5 kg, 71.2 mol) in toluene (431.4 kg), it was washed into the mixture with toluene (12.3 kg) and the mixture was stirred at −5° C. or lower. A mixed solution of p-toluenesulfonyl chloride (28.5 kg, 149 mol, 2.1 eq.) in 4-methyltetrahydropyran (6.1 kg) and toluene (98.6 kg) was added dropwise under a nitrogen atmosphere, and it was washed into the mixture with toluene (24.7 kg) and then it was stirred for 4 hours at −10° C. Upon completion of the reaction, water (159.6 kg) was added dropwise to the reaction mixture prior to liquid separation. After addition of 5 N hydrochloric acid (20.8 kg of 35% hydrochloric acid, 22.3 kg water) to the organic layer and liquid separation, the organic layer was rinsed with water (142.5 kg). After subsequent addition of an aqueous 0.5 N sodium hydroxide solution (2.8 kg caustic soda flake, 142.2 kg water) to the organic layer, and liquid separation, the organic layer was rinsed with water (142.5 kg). The obtained organic layer was concentrated under reduced pressure to 80 L at 50° C. or lower, ethanol (67.5 kg) was added, and the mixture was concentrated under reduced pressure to 80 L. Ethanol (67.3 kg) was added to the concentrate and the mixture was stirred at 0° C. for 2 hours. The precipitate was removed by filtration and washed in with ethanol (67.8 kg), and the filtrate was concentrated under reduced pressure to 80 L. Ethanol (112.4 kg) was added to the obtained concentrate and the mixture was concentrated under reduced pressure to 80 L, to obtain a crude product of the title compound (33.5 kg content as 92%) as an ethanol solution (80 L).

### Production Example 8: Production of 4-{[6-(2-methoxyethoxy)-1H-indol-5-yl]oxy}-N-(2,4,4-trimethylpentan-2-yl)pyridine-2-amine (2f-1)

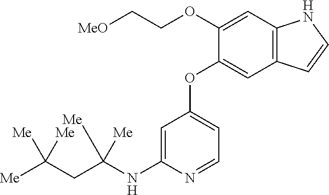 [SMILES: COCCOc1cc2[nH]ccc2cc1Oc1ccnc(NC(C)(C)CC(C)(C)C)c1]

To a solution of 6-(2-methoxyethoxy)-N-methyl-5-({2-[(2,4,4-trimethylpentan-2-yl)amino]pyridin-4-yl}oxy)-1H-indole-1-carboxamide (33.5 kg content, 65.5 mol) in ethanol (80 L) were added ethanol (26.9 kg) and tetrahydrofuran (89.3 kg). An aqueous 5 N sodium hydroxide solution (7.9 kg caustic soda flake, 197 mol, 3.0 eq., 38.2 kg water) was added under a nitrogen atmosphere, and the mixture was stirred at 48° C. for 5 hours. Upon completion of the reaction, the reaction mixture was cooled to 20° C., and methyl tert-butyl ether (173.8 kg), water (115.1 kg) and 5 N hydrochloric acid (15.0 kg of 35% hydrochloric acid, 15.9 kg water) were added prior to liquid separation. Tetrahydrofuran (59.5 kg) and 5% brine (16.8 kg salt, 150.6 kg water) were added to the organic layer prior to liquid separation. The obtained organic layer was concentrated under reduced pressure to 170 L at 50° C. or lower, n-propanol (188.1 kg) was added, and the mixture was concentrated under reduced pressure to 160 L. Methyl tert-butyl ether (49.7 kg) was added to the concentrate and the mixture was stirred for at 48° C. 73 minutes. The suspension was cooled to 0° C. and then filtered and rinsed with n-propanol (53.6 kg). The obtained crystals were dried under reduced pressure at 50° C. or lower to obtain 22.93 kg of the title compound.

¹H NMR Spectrum (CD₃OD) δ (ppm): 0.89 (9H, s), 1.30 (6H, s), 1.64 (2H, s), 3.27 (3H, s), 3.56-3.59 (2H, m), 4.05-4.09 (2H, m), 5.85 (1H, d, J=2.3 Hz), 6.15 (1H, dd, J=6.0, 2.3 Hz), 6.36-6.39 (1H, m), 7.11 (1H, s), 7.17 (1H, d, J=3.4 Hz), 7.25 (1H, s), 7.74 (1H, d, J=6.0 Hz)

### Production Example 9: Production of 6-(2-methoxyethoxy)-N-methyl-5-({2-[(2,4,4-trimethylpentan-2-yl)amino]pyridin-4-yl}oxy)-1H-indole-1-carboxamide (2h-1)

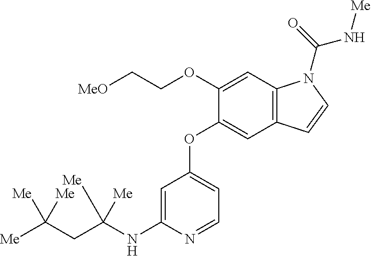 [SMILES: CNC(=O)n1ccc2cc(Oc3ccnc(NC(C)(C)CC(C)(C)C)c3)c(OCCOC)cc21]

Tetrahydrofuran (109.3 kg) was added to 4-{[6-(2-methoxyethoxy)-1H-indol-5-yl]oxy}-N-(2,4,4-trimethylpentan-2-yl)pyridine-2-ami ne (20.5 kg, 49.8 mol), and the mixture was stirred at 5° C. or lower. A solution of potassium tert-butoxide (5.9 kg, 52 mol, 1.05 eq.) in DMSO (22.6 kg) was added dropwise under a nitrogen atmosphere, and the mixture was washed in with DMSO (2.3 kg) and stirred at 0° C. for 30 minutes. A solution of phenylmethyl carbamate (9.0 kg, 60 mol, 1.30 eq.) in tetrahydrofuran (18.2 kg) was added dropwise, and the mixture was washed in with tetrahydrofuran (9.1 kg) and stirred at 0° C. for 5 minutes. Upon completion of the reaction, water (143.5 kg) and isopropyl acetate (125.1 kg) were added to the reaction mixture which was then stirred at 20° C. prior to liquid separation. After addition of an aqueous 1 N sodium hydroxide solution (4.1 kg caustic soda flake, 102.5 kg water) to the organic layer and liquid separation, a 10% aqueous ammonium chloride solution (10.3 kg ammonium chloride, 92.3 kg water) was added prior to liquid separation. The organic layer was rinsed with 1% brine (1.0 kg salt, 102.5 kg water), after which the rinsed organic layer was concentrated under reduced pressure to 80 L at 40° C. or lower, and subjected to azeotropic distillation 3 times with ethanol (80.9 kg), to obtain a crude product of the title compound (23.34 kg content as 100%) as an ethanol solution (80 L).

¹H NMR Spectrum (CD₃OD) δ (ppm): 0.93 (9H, s), 1.35 (6H, s), 1.71 (2H, s), 2.98 (3H, s), 3.30 (3H, s), 3.62-3.64 (2H, m), 4.13-4.16 (2H, m), 5.88 (1H, d, J=2.3 Hz), 6.18 (1H, dd, J=5.9, 2.3 Hz), 6.61 (1H, d, J=3.2 Hz), 7.32 (1H, s), 7.58 (1H, d, J=3.8 Hz), 7.79 (1H, d, J=5.9 Hz), 8.08 (1H, s)

### Production Example 10: Production of 5-((2-aminopyridin-4-yl)oxy)-6-(2-methoxyethoxy)-N-methyl-1H-indole-1-carboxamide methanesulfonate (methanesulfonate of (2i))

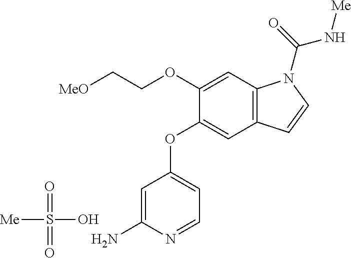

A solution of 6-(2-methoxyethoxy)-N-methyl-5-({2-[(2,4,4-trimethylpentan-2-yl)amino]pyridin-4-yl}oxy)-1H-indole-1-carboxamide (content: 26.0 kg, 55.5 mol) in ethanol (90 L) was concentrated under reduced pressure to 75 L at 40° C. or lower, and then methanol (37.0 kg) and ethanol (17.3 kg) were added. Methanesulfonic acid (42.7 kg, 444 mol, 8.0 eq.) was added at 10° C. under a nitrogen atmosphere, and it was washed into the mixture with methanol (4.2 kg) and stirred at 40° C. for 28 hours. After confirming completion of the reaction, the mixture was cooled to 20° C., methyl tert-butyl ether (385.8 kg) was added dropwise, and stirring was continued for 1 hour. After cooling to 0° C., the mixture was filtered and rinsed with a liquid mixture of methyl tert-butyl ether and ethanol (methyl tert-butyl ether/ethanol=1.64/0.36 vol., 39.2 kg) and isopropyl acetate (45.5 kg). The obtained crystals were dried under reduced pressure at 50° C. or lower to obtain 22.89 kg of the title compound.

¹H NMR Spectrum (DMSO-d₆) δ (ppm): 2.32 (3H, s), 2.84 (3H, d, J-4.5 Hz), 3.15 (3H, s), 3.51-3.54 (2H, m), 4.08-4.11 (2H, m), 6.01 (1H, d, J=2.3 Hz), 6.63-6.66 (2H, m), 7.50 (1H, s), 7.61 (2H, br s), 7.79 (1H, d, J=3.8 Hz), 7.90 (1H, d, J=7.2 Hz), 8.10 (1H, s), 8.18 (1H, q, J-4.2 Hz), 12.82 (1H, brs)

### Production Example 10-2: Production of 5-(2-aminopyridin-4-yl)oxy)-6-(2-methoxyethoxy)-N-methyl-1H-indole-1-carboxamide methanesulfonate (methanesulfonate of (2i))

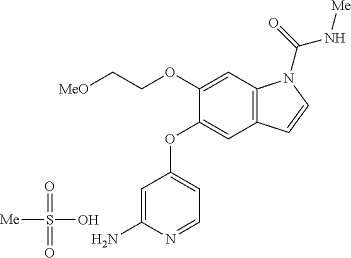

Methanol (33.2 kg) was added to a solution of 6-(2-methoxyethoxy)-N-methyl-5-({2-[(2,4,4-trimethylpentan-2-yl)amino]pyridin-4-yl}oxy)-1H-indole-1-carboxamide (47.8 kg content with previous step as 100%, 102 mol) in ethanol (183 L), under a nitrogen atmosphere. Methanesulfonic acid (117.7 kg, 1225 mol, 12.0 eq.) was added, and it was washed into the mixture with methanol (4.7 kg) and stirred at 25 to 32° C. for 21 hours, after which 5-((2-aminopyridin-4-yl)oxy)-6-(2-methoxyethoxy)-N-methyl-1H-indole-1-carboxamide (46 g) was added. Methyl tert-butyl ether (355.3 kg) was added dropwise to the crystal-precipitated suspension, and the mixture was cooled to 2 to 5° C. The precipitated solid was filtered off and rinsed with a mixture of methyl tert-butyl ether (126.8 kg) and ethanol (54.0 kg), and isopropyl acetate (208.7 kg). The obtained crystals were dried under reduced pressure at 50° C. or lower to obtain 38.04 kg of the title compound.

### Production Example 11: Production of 5-((2-aminopyridin-4-yl)oxy)-6-(2-methoxyethoxy)-N-methyl-1H-indole-1-carboxamide (2i)

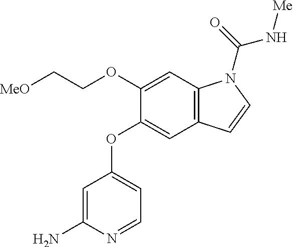 [SMILES: CNC(=O)n1ccc2cc(Oc3ccnc(N)c3)c(OCCOC)cc21]

An aqueous solution of IN sodium hydroxide (2.71 kg caustic soda flake, 67.7 mol, 1.54 eq., 67.7 kg water) was added to a suspension of 5-((2-aminopyridin-4-yl)oxy)-6-(2-methoxyethoxy)-N-methyl-1H-indole-1-carboxamide methanesulfonate (19.9 kg, 44.0 mol) in tetrahydrofuran (159.2 kg) under a nitrogen atmosphere, and the mixture was stirred at 25° C. for 30 minutes. Isopropyl acetate (156.2 kg) was added to the reaction mixture prior to liquid separation, and then the organic layer was rinsed with 5% brine (2.99 kg salt, 56.7 kg water). The obtained organic layer was rinsed with water (59.7 kg) and then clarified by filtration and washed in with isopropyl acetate (8.7 kg). It was subsequently concentrated under reduced pressure to 100 L at 40° C. or lower, and further subjected to azeotropic distillation 4 times with acetonitrile (78.2 kg). Acetonitrile (15.6 kg) was added to the concentrate and the mixture was stirred at 48° C. for 1 hour. The suspension was cooled to 0° C. and then filtered and rinsed with acetonitrile (23.5 kg). The obtained crystals were dried under reduced pressure at 50° C. or lower to obtain 13.91 kg of the title compound.

¹H NMR Spectrum (DMSO-d₆) δ (ppm): 2.83 (3H, d, J-4.4 Hz), 3.18 (3H, s), 3.50-3.54 (2H, m), 4.04-4.08 (2H, m), 5.69 (1H, d, J=1.8 Hz), 5.76 (2H, s), 6.09 (1H, dd, J-5.7, 2.2 Hz), 6.59 (1H, d, J=3.5 Hz), 7.33 (1H, s), 7.71-7.74 (2H, m), 8.03 (1H, s), 8.10-8.14 (1H, m)

### Production Example 11-2: Production of 5-((2-aminopyridin-4-yl)oxy)-6-(2-methoxyethoxy)-N-methyl-1H-indole-1-carboxamide (2i)

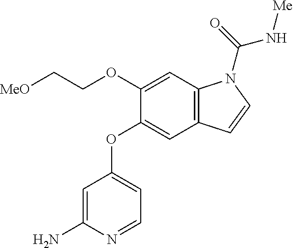 [SMILES: CNC(=O)n1ccc2cc(Oc3ccnc(N)c3)c(OCCOC)cc21]

An aqueous solution of 1 N sodium hydroxide (5.5 kg caustic soda flake, 137.5 mol, 1.70 eq., 138 kg was added water) to a suspension of 5-((2-aminopyridin-4-yl)oxy)-6-(2-methoxyethoxy)-N-methyl-1H-indole-1-carboxamide methanesulfonate (36.6 kg, 80.9 mol) in tetrahydrofuran (292.5 kg) under a nitrogen atmosphere, and the mixture was stirred at 20° C. for 30 minutes. Isopropyl acetate (287 kg) was added to the reaction mixture prior to liquid separation, and then the organic layer was rinsed with 5% brine (5.5 kg salt, 104 kg water). The obtained organic layer was rinsed with water (110 L) and then clarified by filtration and washed in with isopropyl acetate (47.9 kg). It was subsequently concentrated under reduced pressure to 184 L at 40° C. or lower, and further subjected to azeotropic distillation 4 times with acetonitrile (144 kg). Acetonitrile (28.8 kg) was added to the concentrate and the mixture was stirred at 45 to 46° C. for 1 hour. The suspension was cooled to 2° C. and then filtered and rinsed with acetonitrile (43.2 kg). The obtained crystals were dried under reduced pressure at 50° C. or lower to obtain 25.92 kg of the title compound.

1. A process for producing compound (2i): 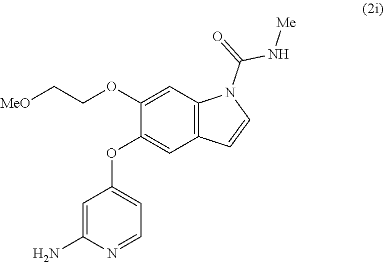 [SMILES: CNC(=O)n1ccc2cc(Oc3ccnc(N)c3)c(OCCOC)cc21]
or a salt thereof, wherein the process comprises:
2-a) step 2-a) in which a protecting group is introduced into compound (1g): 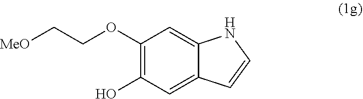 [SMILES: COCCOc1cc2[nH]ccc2cc1O]
to produce compound (2a): 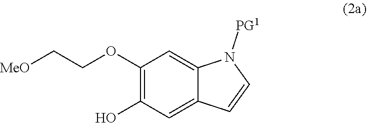
(where PG¹ represents a nitrogen protecting group),
2-b) step 2-b) in which compound (2a) obtained in step 2-a) and compound (2b): 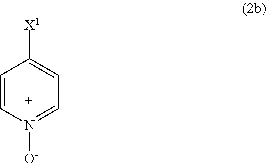
(where X¹ represents a leaving group),
are reacted in the presence of a base to produce compound (2c): 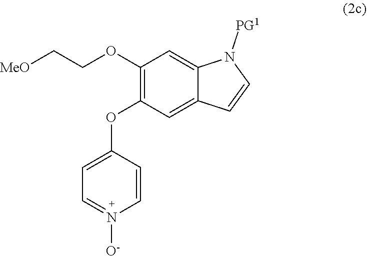
(where PG¹ represents the same group as specified above),
2-c) step 2-c) in which compound (2c) obtained in step 2-b) and compound (2d):
R¹—NH₂ (2d)
(where R¹ represents a tert-pentyl, tert-butyl, tert-octyl or cumyl group),
are reacted in the presence of an activating agent to produce compound (2e): 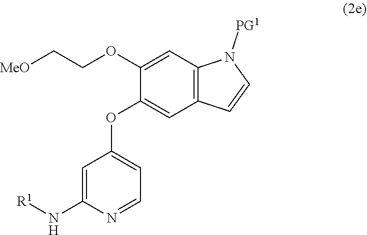
(where PG¹ and R¹ represent the same groups as specified above),
2-d) step 2-d) in which PG¹ in compound (2e) obtained in step 2-c) is removed to produce compound (2f): 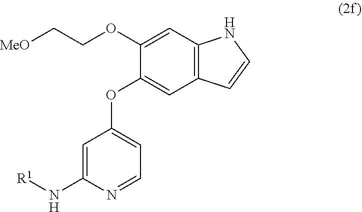
(where R¹ represents the same group as specified above),
2-e) step 2-e) in which compound (2f) obtained in step 2-d) and compound (2g-1) or compound (2g-2): 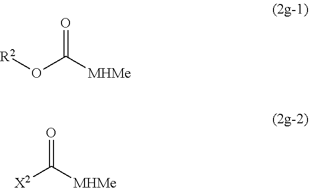
(where R² of formula (2g-1) is a C₁₋₆ alkyl group or C₆₋₁₀ aryl group, the C₁₋₆ alkyl group optionally having 1 to 3 identical or different substituents selected from the group consisting of halogen atoms and methoxy groups, and the C₆₋₁₀ aryl group optionally having 1 to 3 identical or different substituents selected from halogen atoms and methyl, methoxy and nitro groups, and X² of formula (2g-2) represents a halogen atom),
are reacted in the presence of a base to produce compound (2h): 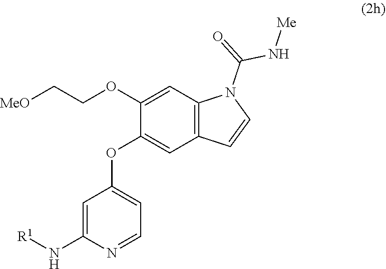
(where R¹ represents the same group as specified above), and
2-f) step 2-f) in which R¹ of compound (2h) obtained in step 2-e) is removed to produce compound (2i): 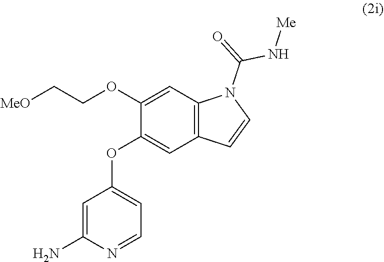 [SMILES: CNC(=O)n1ccc2cc(Oc3ccnc(N)c3)c(OCCOC)cc21]
and if necessary step 2-g) in which compound (2i) obtained in step 2-f) is converted to a salt.

2. The production process according to claim 1, wherein compound (2b) is 4-nitropyridin-1-ium-oleate.

3. The production process according to claim 1, wherein compound (2d) is 1,1,3,3-tetramethylbutylamine.

4. The production process according to claim 1, wherein compound (2d) is cumylamine.

5. The production process according to claim 1, wherein the activating agent is p-toluenesulfonyl chloride.

6. A process for producing compound (1g): 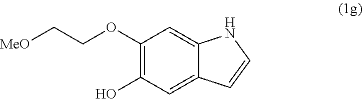 [SMILES: COCCOc1cc2[nH]ccc2cc1O]
wherein the process comprises:
1-a) step 1-a) in which 1,2-(methylenedioxy)-4-nitrobenzene and 4-bromobenzyl alcohol are reacted in the presence of a base to produce compound (1c): 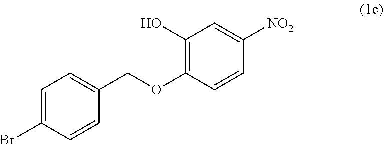 [SMILES: O=[N+]([O-])c1ccc(OCc2ccc(Br)cc2)c(O)c1]
1-b) step 1-b) in which compound (1c) obtained in step 1-a) and a methoxyethylating agent are reacted in the presence of a base to produce compound (1d): 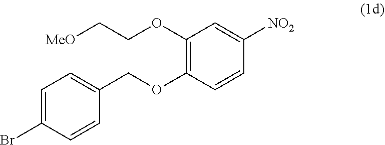 [SMILES: COCCOc1cc([N+](=O)[O-])ccc1OCc1ccc(Br)cc1]
1-c) step 1-c) in which compound (1d) obtained in step 1-b) and a cyanomethylating agent are reacted in the presence of a base to produce compound (1f): 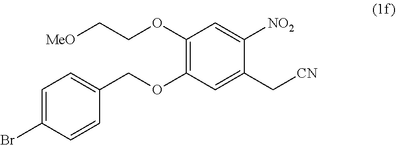 [SMILES: COCCOc1cc([N+](=O)[O-])c(CC#N)cc1OCc1ccc(Br)cc1]
and
1-d) step 1-d) in which compound (1g): 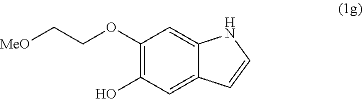 [SMILES: COCCOc1cc2[nH]ccc2cc1O]
is produced by conversion of the nitro group of compound (11) obtained in step 1-c) to an amino group, removal of the 4-bromobenzyl group, and ring closure with an acid catalyst.

7. The production process according to claim 6, wherein a palladium catalyst and sulfuric acid are used in step 1-d).
